(19) United States
(12) Patent Application Publication
(10) Pub. No.: US 2017/0367873 A1
(43) Pub. Date: Dec. 28, 2017

(54) Male Accessory for Disposing Human Waste

(71) Applicant: Dominique Anthony Grannum, Pearland, TX (US)

(72) Inventor: Dominique Anthony Grannum, Pearland, TX (US)

(21) Appl. No.: 15/195,996

(22) Filed: Jun. 28, 2016

(51) Int. Cl. A61F 5/453; A61F 5/44; A41B 9/02

(52) U.S. Cl. CPC A61F 5/453; A41B 9/023; A61F 5/4408

(57) ABSTRACT

A male accessory is disclosed to dispose human waste. The male accessory includes an elastic band, a penile sleeve and a genitalia sleeve. The elastic band may be arranged in a loop to enable the male accessory to be held with the male organ. The penile sleeve disposed within the elastic band. The penile sleeve may be arranged to hold and cover male organ. Further, the genitalia sleeve may be connected to the penile sleeve through a membrane. The genitalia sleeve may be configured to create an inner space to receive the human waste and covers male testicles without any contact thereof with the human waste.

### COPYRIGHT NOTICE

A portion of the disclosure of this patent document contains material which is subject to copyright protection. The copyright owner has no objection to the facsimile reproduction by anyone of the patent document or the patent disclosure, as it appears in the Patent and Trademark Office patent file or records, but otherwise reserves all copyright rights whatsoever. 37 CFR 1.71(d).

### FIELD

The present disclosure generally relates to medical accessories, and, more specifically, to a male accessory for disposal of human waste.

### BACKGROUND

Males having medical problem in defecating the human waste, for example urine, often wear medical devices or undergarments to accept and contain urine. Many of these accessories and undergarments may be an awkward to apply or use. Other accessories are readily apparent to others that may see the device or undergarment, creating embarrassment for the wearer. Still accessories do not provide ease of use when the wearer is using them.

Furthermore, many of the existing accessories and undergarments designed for medical problems involve disposing of the entire device or undergarment after urine is collected within the device or undergarment. In such accessories there may be a contact between the human waste and human skin which leads to rash and chaffing along with the extreme discomfort to human body.

Accordingly, it would be beneficial to provide a male accessory that provides an ease and familiarity of application for the wearer, and appears to others as a typical men's undergarment.

Additionally, it would also be beneficial to provide a male accessory that may be easily used during situations when the user is not able to pass urine in a normal way, increasing the comfort level of such user by helping them to pass their urine in a more effective sanitary process.

### SUMMARY

This Summary is provided to introduce a selection of concepts in a simplified form that are further described below in the Detailed Description. This Summary is not intended to identify key features or essential features of the claimed subject matter, nor it is intended to be used to limit the scope of the claimed subject matter.

In view of the stated drawbacks, the present disclosure discloses a male accessory for disposal of human waste, which will be presented in the following simplified summary to provide a basic understanding of one or more aspects of the disclosure that are intended to overcome the discussed drawbacks, but to include all advantages thereof, along with providing some additional advantages. This summary is not an extensive overview of the disclosure. It is intended to neither identify key or critical elements of the disclosure, nor to delineate the scope of the present disclosure. Rather, the sole purpose of this summary is to present some concepts of the disclosure, its aspects and advantages in a simplified form as a prelude to the more Detailed Description that is presented hereinafter.

An object of the present disclosure is to disclose a male accessory that provides an ease and familiarity of application for the wearer, and appears to others as a typical men's undergarment. Further object is to provide a male accessory that may be easily used during situations when the user is not able to pass urine in a normal way, increasing the comfort level of such user by helping them to pass their urine in a more effective sanitary process. Yet another object of the present disclosure is to prevent contact of human waste with skin of user so that core temperature of the user body is not disturbed by contact of the human waste as well as prevention of skin disease resulted due to contact of the human waste.

In one aspect of the present disclosure a male accessory is disclosed to dispose human waste. The male accessory includes an elastic band, a penile sleeve and a genitalia sleeve. The elastic band may be arranged in a loop to enable the male accessory to be held with the male organ. The penile sleeve disposed within the elastic band. The penile sleeve may be arranged to hold and cover male organ. Further, the genitalia sleeve may be connected to the penile sleeve through a membrane. The genitalia sleeve may be configured to create an inner space to receive the human waste and covers male testicles without any contact thereof with the human waste.

In the present disclosure the membrane may be sealed with the penile sleeve into the genitalia sleeve to prevent the contact of the human waste from skin surface contact of user.

In the present disclosure, the male accessory may further include a pouch connected to the genitalia sleeve to receive and store the human waste. In an embodiment, the pouch may include an absorbent pad placed therein to absorb the human waste. The pouch may be made of an anti-microbial, liquid resistant fabric. The absorbent pad may be selectively removable from the pouch. The penile sleeve is configured to pass the human waste to the genitalia sleeve.

In another aspect of the present disclosure a wearable male garment for collecting and disposing human waste is disclosed. The wearable male garment includes an undergarment having an interior space; and a male accessory removable coupled to the undergarment along the interior space. The male accessory having, an elastic band arranged in a loop to hold the male accessory to the male organ, a penile sleeve disposed within the elastic band and arranged to hold and cover the male organ, and, a genitalia sleeve connected to the penile sleeve through a membrane. The genitalia sleeve may be configured to create an inner space to receive the human waste and covers male testicles without any contact thereof with the human waste.

These together with the other aspects of the present disclosure, along with the various features of novelty that characterize the present disclosure, are pointed out with particularity in the present disclosure. For a better understanding of the present disclosure, its operating advantages, and its uses, reference should be made to the accompanying drawings and descriptive matter in which there are illustrated exemplary embodiments of the present disclosure.

### BRIEF DESCRIPTION OF THE DRAWINGS

The foregoing summary, as well as the following Detailed Description of preferred embodiments, is better understood when read in conjunction with the appended drawings. For the purposes of illustration, there is shown in the drawings exemplary embodiments; however, the present disclosure is not limited to the specific methods and instrumentalities disclosed. In the drawings:

FIG. 1A illustrates a side view of a male accessory accommodated in a wearable garment, in accordance with an exemplary embodiment of the present disclosure; and

FIG. 1B illustrates perspective view of a male accessory accommodated in a wearable garment, in accordance with an exemplary embodiment of the present disclosure.

### DETAILED DESCRIPTION

The following description and drawings are illustrative and are not to be construed as limiting. Numerous specific details are described to provide a thorough understanding. However, in certain instances, well known or conventional details are not described in order to avoid obscuring the description. References to one or an embodiment in the present disclosure are not necessarily references to the same embodiment; and, such references mean at least one.

Reference in this specification to “one embodiment” or “an embodiment” means that a particular feature, structure, or characteristic described in connection with the embodiment is included in at least one embodiment of the disclosure. The appearances of the phrase “in one embodiment” in various places in the specification are not necessarily all referring to the same embodiment, nor are separate or alternative embodiments mutually exclusive of other embodiments. Moreover, various features are described which may be exhibited by some embodiments and not by others. Similarly, various requirements are described which may be requirements for some embodiments but not other embodiments.

Reference throughout this specification to “a select embodiment,” “one embodiment,” or “an embodiment” means that a particular feature, structure, or characteristic described in connection with the embodiment is included in at least one embodiment of the disclosed subject matter. Thus, appearances of the phrases “a select embodiment,” “in one embodiment,” or “in an embodiment” in various places throughout this specification are not necessarily referring to the same embodiment.

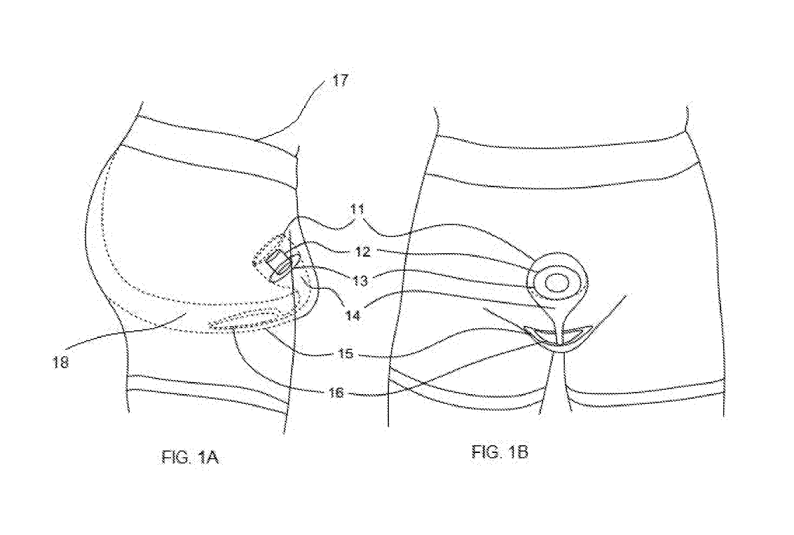

Referring to FIGS. 1A and 1B, side and front views of a male accessory 10 accommodated in a male wearable garment are respectively depicted, in accordance with an exemplary embodiment of the present disclosure. As also shown in FIGS. 1A and 1B, the male accessory 10 may include an elastic band 11, a penile sleeve 12 and a genitalia sleeve 14 seamlessly configured to form a sleeve like configuration capable of collecting and disposing human waste. Such sleeve like configuration of the male accessory 10 may be made of latex and placed around the entire male genitalia through an elastic band 11. The elastic band 11 arranged in a loop to enable the male accessory 10 to be held around male genitalia/organ. The elastic band 11 may be a non-restrictive elastic band. However, without departing from the scope of present disclosure, the elastic band 11 may be made of any other elastic material that may held the male accessory 10 around the male genitalia.

The penile sleeve 12 of male accessory 10 may be disposed along the elastic band 11 and arranged to hold and cover male organ. In one example, the male organ may be placed entirely in penile sleeve 12 such that the elastic band 11 may be located at the base of the male organ, i.e., where the male organ meets the body of the wearer. As described above the penile sleeve 12 may be made of latex material. Such penile sleeve 12 of latex material may have reasonable elasticity to accommodate various shapes and sizes of male organ. The penile sleeve 12 may include, but not limiting to, cylindrical shape to correspond and accommodate the shapes and sizes of male organ.

The genitalia sleeve 14 may be connected to the penile sleeve 12 through a membrane 13. The genitalia sleeve 14 may be configured to create an inner space 18 to receive the human waste and covers male testicles without any contact thereof with the human waste. Specifically, the genitalia sleeve 14 is connected to the penile sleeve 12 through the membrane 13 in the male accessory 10 to cover male testicles thus covering the remaining part of the male genitalia apart from the male organ. The membrane 13 may sealed the penile sleeve 12 into the genitalia sleeve 14 to prevent the contact of the human waste from skin of the user. Such genitalia sleeve 14 of latex material may have reasonable elasticity to accommodate various shapes and sizes of male testicles. Further, the genitalia sleeve 14 may also correspond to a shape similar to the shape of the male testicles.

The genitalia sleeve 14 may be connected with a pouch 15. The pouch 15 may be accommodated in the genitalia sleeve 14, which may receive and store the human waste without any contact of the human waste from skin of the user. The pouch 15 may include an absorbent pad 16 placed inside the pouch 15. The absorbent pad 16 includes a material that may be capable of absorbing moisture. For the purposes of this disclosure, a material that contains moisture can be defined as a moisture absorbent material that may hold moisture such as human urine within the confines of the material to eliminate contact of expelled urine with the wearer's skin and garments. In a further example, the moisture absorbent material can be composed of numerous types of materials including, but not limited to, cellulosic fluff pulp, tissue layers, air-laid materials, highly absorbent polymers, absorbent foam materials, absorbent non-woven materials or the like. In one example, the absorbent pad 16 may absorb several times its own weight in liquid (e.g., urine). Additionally, the absorbent pad 16 may be designed to contain at least the average amount of incontinent urine from an adult male. In a more particular example, the absorbent pad 16 may be designed to contain at least the average amount plus a predetermined amount of incontinent urine from an adult male. The absorbent pad 16 may be selectively removable from the pouch 15.

The male accessory 10 prevents any type of contact at any stage of time in any way from human waste with human skin of the user. The pouch 15 includes an antimicrobial, liquid resistant fabric. The anti-microbial, liquid resistant fabric faces the interior of the pouch 15. The anti-microbial, liquid resistant fabric promote sanitary health and limit or eliminate passage of urine from the pouch 15 to undesired locations. The combination of the moisture absorbent material in the pad 16 and the anti-microbial, liquid resistant fabric in the pouch 15 may limit or eliminate liquid and/or moisture leakage from the pouch 15 to undesired locations. In fact, the pad 16 can be an autonomous, stand-alone moisture containing environment to accommodate the male organ of the wearer and contain any moisture from urine leakage. The human waste is prevented from contacting the skin of the user by containing it within the pad 16 or alternately, within the pad 16 and the pouch 15.

In one embodiment of the present disclosure, a wearable male garment for collecting and disposing human waste may be provided. The wearable male garment may be an undergarment 17 defining an interior space. A male accessory, such as the male accessory 10, for disposing human waste may be placed in the interior space and secured to the undergarment 17. The male accessory 10 may be removably placed in the interior space of the undergarment 17 to be easily removed from the undergarment for change over or cleaning of the undergarments 17. The description of the male accessory is not repeated herein for the sake of brevity.

The undergarment 17 is shown in a form similar to undergarments known as boxer, this depiction is not meant to be limiting on the present disclosure, and the undergarment can be similar to other types of undergarments such as no-fly briefs, fly-front briefs, boxer-briefs, etc. As with typical designs of human undergarments, the undergarment 17 can be designed and/or tailored to provide a particular level of comfort and allow a particular range of motion for the wearer of the undergarment 17. In one example, the undergarment 17 can be designed to aesthetically appear as a typical pair of men's underwear, such that a casual observer of the undergarment 17 would not be able to differentiate the undergarment 17 from a typical pair of men's underwear. Additionally, the undergarment 17 can be constructed of materials that are standard materials in men's underwear. Designs such as these can help to alleviate and/or eliminate any embarrassment that the wearer of the male accessory 10 may experience in situations when the male accessory 10 is visible to others. In one example, the undergarment 17 is reusable and washable, enabling the undergarment 17 to be laundered as a typical pair of men's underwear.

Advantageously the present male accessory 10 prevents contact of the user human waste for example urine with the contact of the skin of the user, thereby negating conditions to affect one's core body temperature by urine which has lost heat because it is no longer within the body and prevent the conditions for hypothermia. It can be used for longer hours for example can be used in night time, allowing urinating without disturbing sleep and can be in difficult situations. The present male accessory is easy and simple to use. All males including but not limited to babies, adolescent males, adult males, males in space, males in military, males with special needs can use this easily and safely. The present male accessory can be used in both wet and dry conditions, under high temperature and pressure, in light and dark.

The foregoing descriptions of specific embodiments of the present disclosure have been presented for purposes of illustration and description. They are not intended to be exhaustive or to limit the present disclosure to the precise forms disclosed, and obviously many modifications and variations are possible in light of the above teaching. The embodiments were chosen and described in order to best explain the principles of the present disclosure and its practical application, to thereby enable others skilled in the art to best utilize the present disclosure and various embodiments with various modifications as are suited to the particular use contemplated. It is understood that various omission and substitutions of equivalents are contemplated as circumstance may suggest or render expedient, but such are intended to cover the application or implementation without departing from the spirit or scope of the claims of the present disclosure.

1. A male accessory to be worn around a male organ to collect and dispose human waste, the male accessory comprising:
an elastic band arranged in a loop to held the male accessory to the male organ;
a penile sleeve disposed within the elastic band and arranged to hold and cover the male organ; and
a genitalia sleeve connected to the penile sleeve through a membrane, the genitalia sleeve configured to create an inner space to receive the human waste and covers male testicles without any contact thereof with the human waste.

2. The male accessory according to claim 1 further comprising a pouch connected to the genitalia sleeve to receive and store the human waste.

3. The male accessory according to claim 2, wherein the pouch is of an antimicrobial, liquid resistant fabric material.

4. The male accessory according to claim 2 further comprising an absorbent pad placed inside pouch to absorb the human waste.

5. The male accessory according to claim 4, wherein the absorbent pad is selectively removable from the pouch.

6. The male accessory according to claim 1, wherein the elastic band is a non-restrictive elastic band.

7. The male accessory according to claim 1, wherein the penile sleeve is configured to pass the human waste to the genitalia sleeve.

8. The male accessory according to claim 1, wherein the membrane is sealed the penile sleeve into the genitalia sleeve to prevent the contact of the human waste from skin surface contact of user.

9. The male accessory according to claim 1, wherein the male accessory is placed over entire male genitalia.

10. A wearable male garment for collecting and disposing human waste, the wearable male garment comprising:
an undergarment having an interior space; and
a male accessory removably coupled to the undergarment along the interior space, the male accessory having,
an elastic band arranged in a loop to held the male accessory to the male organ,
a penile sleeve disposed within the elastic band and arranged to hold and cover the male organ, and
a genitalia sleeve connected to the penile sleeve through a membrane, the genitalia sleeve configured to create an inner space to receive the human waste and covers male testicles without any contact thereof with the human waste.
